(19) United States
(12) Patent Application Publication
(10) Pub. No.: US 2017/0003241 A1
(43) Pub. Date: Jan. 5, 2017

(54) SENSOR DEVICE

(71) Applicant: AISAN KOGYO KABUSHIKI KAISHA, Obu-shi (JP)

(72) Inventor: Nobuhiro KATO, Tokai-shi (JP)

(21) Appl. No.: 15/100,112

(22) PCT Filed: Sep. 16, 2014
(86) PCT No.: PCT/JP2014/074419

§ 371 Date: May 27, 2016

(30) Foreign Application Priority Data
Dec. 9, 2013 (JP) 2013-254321

(51) Int. Cl. G01N 27/22; F02M 37/00; F02M 37/22; F02M 37/04

(52) U.S. Cl. CPC G01N 27/226; F02M 37/04; F02M 37/0047; F02M 37/22; G01N 33/22

(57) ABSTRACT

A sensor device 1 includes a fuel passage through which fuel ejected from a fuel pump 4 in a fuel tank 2 flows; and detection electrodes 61 configured to make contact with fuel flowing through the fuel passage to detect a concentration of alcohol contained in the fuel (a first property of the fuel), and a throttle 25 configured to resist a flow of the fuel on a downstream side of the detection electrodes 61 is provided in the fuel passage.

### TECHNICAL FIELD

The present teachings relate to a sensor device.

### BACKGROUND ART

Patent Literature 1 (Japanese Patent Application Publication No. 2012-108030) discloses a sensor device that detects a concentration of alcohol contained in fuel. The sensor device includes a fuel pump that ejects fuel and a fuel property sensor that detects a property of the fuel. The fuel ejected from the fuel pump is sent to the fuel property sensor, and the concentration of alcohol contained in the fuel is detected.

### SUMMARY OF INVENTION

### Technical Problem

The fuel ejected from the fuel pump is ejected under high pressure. The fuel ejected under high pressure may generate bubbles (suffer from cavitation) by having its pressure reduced during a course of being sent to the fuel property sensor. It is therefore an object of the present teachings to provide a sensor device that makes it possible to suppress bubbles from being generated in fuel.

### Solution to Technical Problem

A sensor device disclosed herein comprises: a fuel passage through which fuel ejected from a fuel pump in a fuel tank flows; and a detection electrode configured to make contact with fuel flowing through the fuel passage to detect a first property of fuel. A first resistance portion configured to resist a flow of fuel on a downstream side of the detection electrode is provided in the fuel passage.

With such a configuration, the flow of fuel can be obstructed by the first resistance portion. This makes it possible to suppress a reduction in pressure of the fuel flowing through the fuel passage on an upstream side of the first resistance portion. Therefore, the generation of bubbles in the fuel (cavitation) can be suppressed, as the pressure of the fuel flowing through the fuel passage is not reduced.

### BRIEF DESCRIPTION OF DRAWINGS

FIG. 1 is a schematic view of a configuration of a sensor device according to an embodiment;

FIG. 2 is an enlarged cross-sectional view showing main components of the sensor device according to the embodiment;

FIG. 3 is an enlarged cross-sectional view showing main components of a sensor device according to another embodiment;

FIG. 4 is an enlarged cross-sectional view showing main components of a sensor device according to still another embodiment;

FIG. 5 is a schematic view of a configuration of a sensor device according to still another embodiment;

FIG. 6 is a schematic view of a configuration of a sensor device according to still another embodiment;

FIG. 7 is a schematic view of a configuration of a sensor device according to still another embodiment;

FIG. 8 is a schematic view of a configuration of a sensor device according to still another embodiment;

FIG. 9 is a schematic view of a configuration of a sensor device according to still another embodiment;

FIG. 10 is an enlarged cross-sectional view showing main components of a sensor device according to still another embodiment;

FIG. 11 is an enlarged cross-sectional view showing main components of a sensor device according to still another embodiment; and

FIG. 12 is a schematic view of a configuration of a sensor device according to still another embodiment.

### DESCRIPTION OF EMBODIMENTS

Some of the features characteristic to below-described embodiments will herein be listed. It should be noted that the respective technical elements are independent of one another, and are useful solely or in combinations. The combinations thereof are not limited to those described in the claims as originally filed.

(Feature 1) A second resistance portion configured to resist the flow of fuel on an upstream side of the detection electrode may be provided in the fuel passage, and a resistance of the first resistance portion to the flow of fuel may be greater than a resistance of the second resistance portion to the flow of fuel. With this configuration, a reduction in pressure of fuel near the detection electrode can be suppressed by the first resistance portion and the second resistance portion. This makes it possible to suppress the generation of bubbles in the fuel near the detection electrode.

(Feature 2) The sensor device may further comprise: a set plate attached to the fuel tank; a first case housing the detection electrode and forming a part of the fuel passage; and a cover case formed integrally with the set plate, and covering the first case. A first discharge port configured to discharge fuel in the first case to outside may be provided in the first case, a second discharge port communicating with the first discharge port of the first case may be provided in the cover case, and the first resistance portion may be formed by the second discharge port.

(Feature 3) The sensor device may further comprise: a set plate attached to the fuel tank; and a first case housing the detection electrode and forming a part of the fuel passage. A discharge port configured to discharge fuel in the first case to outside may be provided in the first case, and the first resistance portion may be formed by the discharge port. With this configuration, it is not necessary to separately provide a member for forming the first resistance portion, as the first resistance portion is formed by the discharge port. This makes it possible to reduce a number of components of the sensor device.

(Feature 4) The sensor device may further comprise a second case configured to store fuel therein to detect a second property of fuel. The fuel passage may communicate with an inside of the second case on a downstream side of the detection electrode. With this configuration, the fuel can be sent to the second case in a state where a flow velocity of the fuel has been increased by the first resistance portion. This makes it possible to easily transport the fuel to the second case.

(Feature 5) An introduction port configured to introduce fuel into the second case may be provided in the second case of the sensor device, and the first resistance portion may be formed by the introduction port. This configuration makes it possible to increase the flow velocity of the fuel by the introduction port arranged in the second case, thus making it possible to easily transport the fuel to the second case.

(Feature 6) The sensor device may further comprise: a second case configured to store fuel therein to detect a second property of fuel; and a branch passage branching from the fuel passage on an upstream side of the detection electrode, and communicating with an inside of the second case. With this configuration, a portion of the fuel flowing through the fuel passage can be transported to the second case through the branch passage. This makes it possible to adjust a flow volume of the fuel flowing through the fuel passage.

(Feature 7) An introduction port configured to introduce fuel into the second case may be provided in the second case of the sensor device, and a third resistance portion may be formed by the introduction port. With this configuration, the pressure of fuel that is introduced from the branch passage into the second case can be adjusted by the third resistance portion. This makes it possible to adjust the flow quantities of the fuel flowing through the fuel passage and the branch passage.

(Feature 8) The sensor device may further comprise a relief mechanism configured to allow fuel to flow out of the fuel passage on an upstream side of the detection electrode. With this configuration, the flow volume of fuel that is sent to the detection electrode can be held constant by the relief mechanism allowing the fuel to flow out.

(Feature 9) The first resistance portion may be formed by a throttle or a filter arranged in the fuel passage.

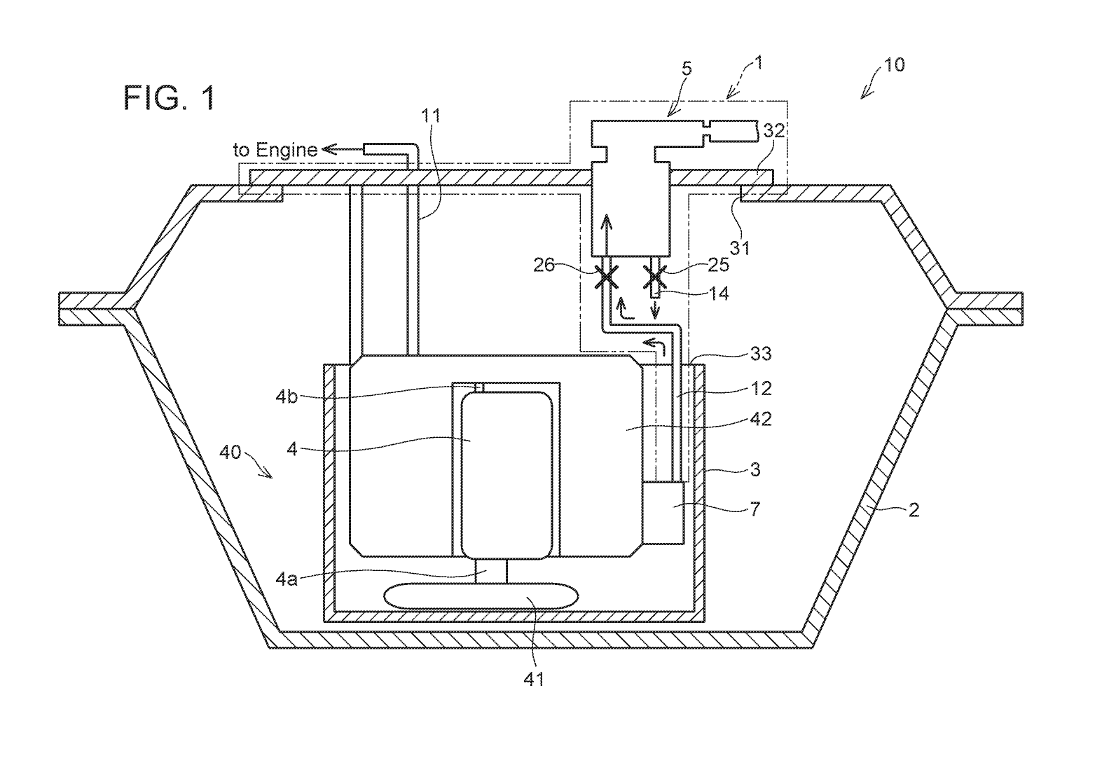

Embodiments will be described below with reference to the accompanying drawings. As shown in FIG. 1, a fuel supply unit 10 according to an embodiment comprises a fuel tank 2 that houses fuel and a sensor device 1 attached to the fuel tank 2. The fuel supply unit 10 supplies fuel to an engine of a vehicle. Further, the sensor device 1 detects a property of the fuel. More specifically, the sensor device 1 detects a concentration of alcohol contained in the fuel.

Fuel is stored in the fuel tank 2. The fuel contains gasoline and alcohol. Further, a pump unit 40 is disposed in the fuel tank 2. An opening 31 is provided in an upper part of the fuel tank 2.

The pump unit 40 comprises a reserve cup 3, a fuel pump 4, a suction filter 41, a high-pressure filter 42, a pressure regulator 7, and a supply line 11.

The reserve cup 3 is disposed in a bottom part of the fuel tank 2. The reserve cup 3 has an opening 33, and is disposed with the opening 33 facing upward. A portion of the fuel housed in the fuel tank 2 is stored in the reserve cup 3. Further, the fuel pump 4 is disposed in the reserve cup 3.

The fuel pump 4 sucks the fuel housed in the reserve cup 3, pressurizes the fuel thus sucked, and ejects the fuel thus pressurized. The fuel is ejected from the fuel pump 4 under high pressure. The fuel pump 4 is connected to an ECU (engine control unit; not illustrated) and driven under a control of the ECU.

The fuel pump 4 has a suction port 4a to which the suction filter 41 is attached. The suction filter 41 removes foreign matter from the fuel when the fuel pump 4 sucks the fuel. The fuel pump 4 has an ejection port 4b to which the high-pressure filter 42 is attached. The high-pressure filter 42 removes foreign matter from the fuel when the fuel pump 4 ejects the fuel.

The pressure regulator 7 is connected to the fuel pump 4 via the high-pressure filter 42. The pressure regulator 7 adjusts the pressure of fuel ejected from the fuel pump 4. The fuel pressurized by the fuel pump 4 flows into the pressure regulator 7. The pressure regulator 7 adjusts the fuel pressure by ejecting into the reserve cup 3 a portion of the fuel having flowed into the pressure regulator 7.

One end of the supply line 11 is connected to the fuel pump 4 via the high-pressure filter 42. The other end of the supply line 11 is connected to the engine of the vehicle. The fuel ejected from the fuel pump 4 flows into the supply line 11 under high pressure. The fuel is sent to the engine of the vehicle through the supply line 11.

The sensor device 1 comprises a set plate 32 that is attached to the fuel tank 2 and a fuel property sensor 5 attached to the set plate 32. Further, the sensor device 1 comprises a guiding line 12 and a discharge line 14. Further, the sensor device 1 comprises a first throttle 25 (which is an example of a first resistance portion) provided in the discharge line 14 and a second throttle 26 (which is an example of a second resistance portion) provided in the guiding line 12.

The set plate 32 is fixed to the upper part of the fuel tank 2 and closes the opening 31 of the fuel tank 2.

The fuel property sensor 5 is fixed to the set plate 32. The fuel property sensor 5 is a sensor configured to be capable of detecting a property of the fuel. More specifically, the fuel property sensor 5 is a sensor configured to detect a concentration of alcohol contained in the fuel (which is an example of a first property). A usable example of the fuel property sensor 5 is a capacitive sensor configured to output a capacitance corresponding to a relative dielectric constant of the fuel as a signal corresponding to the concentration of alcohol.

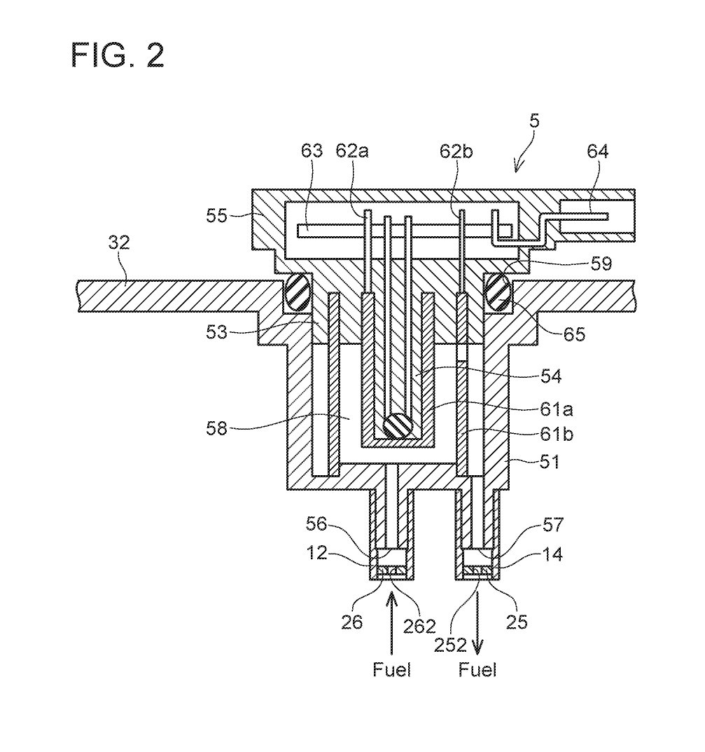

The fuel property sensor 5 is not limited to any particular configuration. However, in the present embodiment, as shown in FIG. 2, the fuel property sensor 5 comprises a lower case 51 (which is an example of a first case) and an upper case 55. The lower case 51 (which is an example of the first case) houses a pair of detection electrodes 61 (inner electrode 61a, outer electrode 61b), and the upper case 55 houses a circuit portion 63. The pair of detection electrodes 61 (inner electrode 61a, outer electrode 61b) and the circuit portion 63 are electrically connected to each other via a pair of internal terminals 62a and 62b. Further, an external terminal 64 is electrically connected to the circuit portion 63. The circuit portion 63 processes electrical signals that are input from the internal terminals 62a and 62b, and outputs the electrical signals thus processed to an external circuit via the external terminal 64. The inner electrode 61a and the outer electrode 61b are in a shape of cylinders. The inner electrode 61a is housed on an inner side of the outer electrode 61b. The outer electrode 61b surrounds a periphery of the inner electrode 61a.

The lower case 51 is attached to the set plate 32. The lower case 51 has an opening 59 formed therein. Further, the outer electrode 61b is disposed in the lower case 51. The outer electrode 61b is in contact with a bottom part of the lower case 51 and extends between the lower case 51 and the upper case 55 in an up and down direction. Further, the lower case 51 has an introduction port 56 and a discharge port 57 arranged in the bottom part thereof. The guiding line 12 is connected to the introduction port 56 so that the fuel is introduced from the guiding line 12 into the lower case 51 via the introduction port 56. The discharge line 14 is connected to the discharge port 57 so that the fuel is discharged from the lower case 51 into the discharge line 14 via the discharge port 57.

A lid portion 53 is fixed to the upper case 55. The lid portion 53 closes the opening 59 of the lower case 51. A sealing material 65 is disposed between the lower case 51 and the lid portion 53. The sealing material 65 tightly closes a gap between the upper case 51 and the lid portion 53. The lid portion 53 has a protruding portion 54 formed thereon. The protruding portion 54 extends downward. The inner electrode 61a is fixed to the protruding portion 54. Further, the outer electrode 61b is fixed to the lid portion 53 in such a way as to surround the inner electrode 61a. A surface of the inner electrode 61a faces a surface of the outer electrode 61b.

A space surrounded by the lower case 51, the lid portion 53, the inner electrode 61a, and the outer electrode 61b forms a housing space 58 capable of housing fuel whose property is to be detected. Fuel introduced into the lower case 51 from the introduction port 56 flows through the housing space 58 to be discharged out of the lower case 51 from the discharge port 57.

The pair of detection electrodes 61 (inner electrode 61a, outer electrode 61b) is a component configured to detect the capacitance of fuel. The pair of detection electrodes 61 (inner electrode 61a, outer electrode 61b) faces the housing space 58 and is configured to make contact with the fuel flowing through the housing space 58.

As shown in FIG. 1, the guiding line 12 has one end connected to the pressure regulator 7 and the other end connected to the fuel property sensor 5. Fuel ejected from the fuel pump 4 flows into the guiding line 12 via the high-pressure filter 42 and the pressure regulator 7. The guiding line 12 guides, to the fuel property sensor 5, the fuel having passed through the pressure regulator 7. This causes the fuel ejected from the fuel pump 4 to be guided to the fuel property sensor 5 by the guiding line 12. The fuel having flowed through the guiding line 12 is introduced into the fuel property sensor 5.

The discharge line 14 has one end connected to the fuel property sensor 5 and the other end opening toward an inside of the reserve cup 3. Fuel discharged from the fuel property sensor 5 flows into the discharge line 14. The fuel having flowed through the discharge line 14 flows into the reserve cup 3.

An inside of the guiding line 12, an inside of the discharge line 14, and the housing space 58 form a fuel passage through which fuel flows. The fuel ejected from the fuel pump 4 flows through the fuel passage. The detection electrodes 61 are configured to make contact with the fuel flowing through the fuel passage. The fuel property sensor 5 is configured to detect a property of fuel via the detection electrodes 61 during a course of the fuel flowing through the fuel passage.

The throttle 25 (which is an example of the first resistance portion) is disposed in vicinity of the fuel property sensor 5. As shown in FIG. 2, the throttle 25 is disposed in the discharge line 14. The throttle 25 is formed in a ring shape. An outer circumferential surface of the throttle 25 is in close contact with an inner circumferential surface of the discharge line 14. The throttle 25 has a communication hole 252 formed at a central part thereof. Upstream and downstream sides of the throttle 25 communicate with each other via the communication hole 252. The communication hole 252 is smaller in diameter than the discharge line 14 (that is, the communication hole 252 is smaller in cross-sectional area than the discharge line 14). By constricting the flow passage of fuel (making the flow passage of fuel smaller), the throttle 25 is configured to resist the flow of fuel through the discharge line 14.

The throttle 26 has a communication hole 262 formed at a central part thereof. A description of the throttle 26 (which is an example of a second resistance portion) is omitted, as the throttle 26 has the same configuration as the throttle 25 (which is an example of the first resistance portion) disposed in the discharge line 14, except that the throttle 26 is disposed in the guiding line 12.

The communication hole 252 of the throttle 25 (which is an example of the first resistance portion) is smaller in diameter than the communication hole 262 of the throttle 26 (which is an example of the second resistance portion) (that is, the communication hole 252 of the throttle 25 is smaller in cross-sectional area than the communication hole 262 of the throttle 26). Therefore, the resistance of the throttle 25 to the flow of fuel is greater than the resistance of the throttle 26 to the flow of fuel (that is, the resistance of the throttle 26 is smaller than the resistance of the throttle 25).

A comparison between magnitudes of resistance of the throttles 25 and 26 can be made on a basis of flow volumes of fuel that passes through the throttles 25 and 26. For example, in a case where the flow volumes of fuel that flows into the throttles 25 and 26 are equal, the magnitudes of resistance can be compared on the basis of the quantities of flow of fuel that flows out from the throttles 25 and 26. In the present embodiment, the flow volumes of fuel that flows into the throttles 25 and 26 are equal, and the flow volume of fuel that flows out from the throttle 25 is smaller than the flow volume of fuel that flows out from the throttle 26. Therefore, the resistance of the throttle 25 (which is an example of the first resistance portion) is greater than the resistance of the throttle 26 (which is an example of the second resistance portion). It should be noted that a comparison between the magnitudes of resistance of the throttles 25 and 26 can be made by other parameter than the flow volume of fuel.

The following will describe an operation of the sensor device configured as above mentioned. In the sensor device 1, when the fuel pump 4 ejects the fuel within the reserve cup 3, the fuel thus ejected flows through the supply line 11 to be sent to the engine. Further, the fuel ejected from the fuel pump 4 is sent to the pressure regulator 7, has its pressure adjusted by the pressure regulator 7, and then is sent to the guiding line 12. The fuel sent to the guiding line 12 flows through the guiding line 12 to be sent to the fuel property sensor 5. Then, the concentration of alcohol contained in the fuel is detected by the fuel property sensor 5. Further, the fuel whose concentration has been detected is discharged into the discharge line 14, and flows through the discharge line 14 to be returned into the reserve cup 3. When the fuel flows through the discharge line 14, the throttle 25 obstructs the flow of the fuel.

As is evident from the foregoing descriptions, with the sensor device 1 according to the embodiment, in which the throttle 25 configured to resist the flow of fuel on a downstream side of the detection electrodes 61 is provided, the flow of fuel can be obstructed by the throttle 25. This makes it possible to suppress a reduction in pressure of the fuel flowing through the fuel passage on an upstream side of the throttle 25. The generation of bubbles in the fuel (cavitation) can be suppressed, as the pressure of the fuel flowing through the fuel passage is not reduced. That is, while the reduction in pressure of the fuel flowing through the fuel passage causes bubbles to be generated in the fuel, the throttle 25 suppresses the reduction in pressure of the fuel and therefore makes it possible to suppress the bubbles from being generated in the fuel.

While one embodiment has been described above, a specific aspect is not limited to the embodiment. For example, while the throttle 26 was disposed in the guiding line 12 in the embodiment, without being limited to this configuration, the throttle 26 may be omitted from the guiding line 12 to.

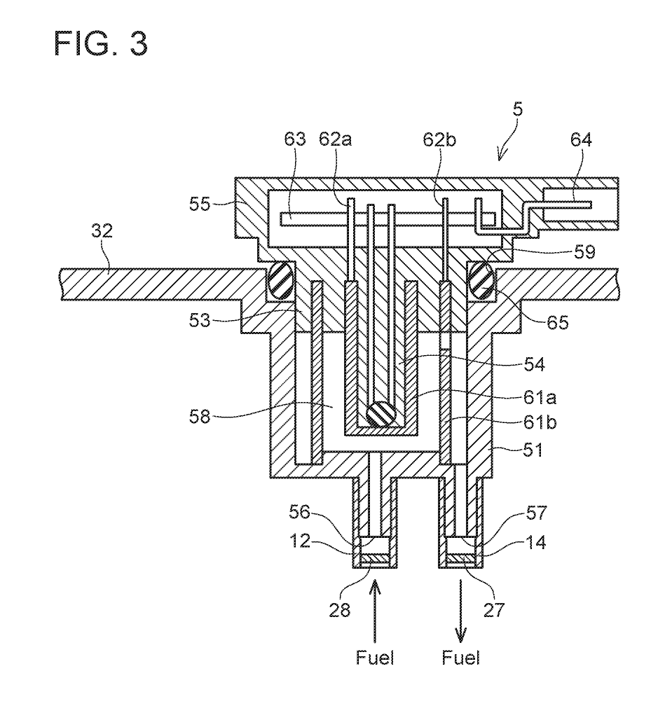

Further, while the first and second resistance portions are configured by the throttles 25 and 26, respectively, the first and second resistance portions are not limited in configuration to the embodiment and may both be configured by filters. Alternatively, either the first or second resistance portion may be configured by a filter. In a sensor device 1 according to another embodiment, as shown in FIG. 3, a filter 27 (which is another example of the first resistance portion) is disposed in the discharge line 14. Further, a filter 28 (which is another example of the second resistance portion) is disposed in the guiding line 12. The filters 27 and 28 are in mesh form and obstruct the flow of fuel. The resistance of the filter 27 to the flow of fuel is greater than the resistance of the filter 28 to the flow of fuel. The magnitudes of resistance to the flow of fuel can be adjusted by adjusting mesh roughness of the filters 27 and 28.

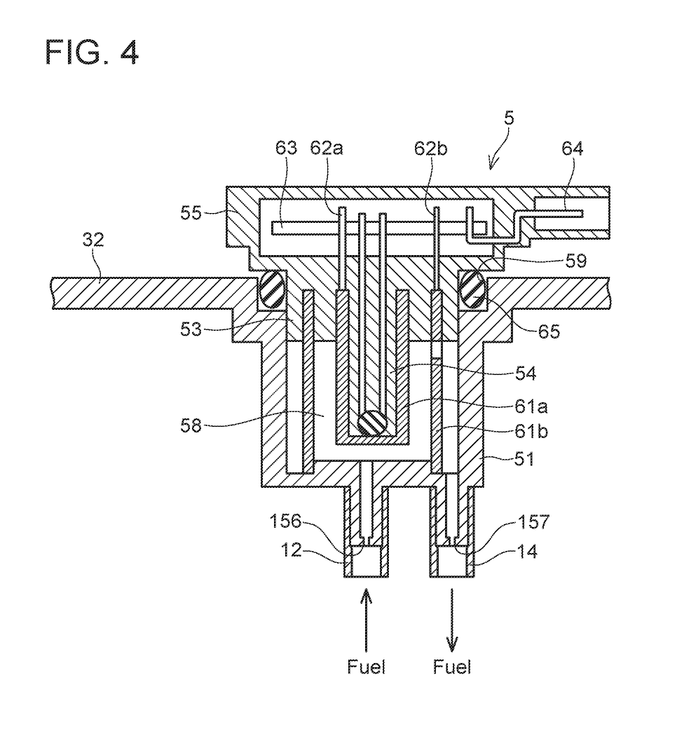

Further, while the first throttle 25 (which is an example of the first resistance portion) was provided in the discharge line 14 in the embodiment, the first throttle 25 is not limited to this configuration. In a sensor device 1 according to another embodiment, as shown in FIG. 4, a first throttle (which is an example of the first resistance portion) is formed by a discharge port 157 arranged in the lower case 51. The area of the flow passage at the discharge port 157 is smaller than the flow passage areas of the fuel passage on upstream and downstream sides of the discharge port 157. In other words, a diameter of the discharge port 157 is smaller than diameters of the fuel passage on the upstream and downstream sides of the discharge port 157. The flow of fuel is obstructed by the discharge port 157 (first throttle).

Further, while the second throttle 26 (which is an example of the second resistance portion) was provided in the guiding line 12 in the embodiment, the second throttle 26 is not limited to this configuration. In the sensor device 1 shown in FIG. 4, a second throttle (which is an example of the second resistance portion) is formed by an introduction port 156 arranged in the lower case 51. The area of the flow passage at the introduction port 156 is smaller than the flow passage areas of the fuel passage on upstream and downstream sides of the introduction port 156. In other words, a diameter of the introduction port 156 is smaller than diameters of the fuel passage on the upstream and downstream sides of the introduction port 156. The flow of fuel is obstructed by the introduction port 156 (second throttle).

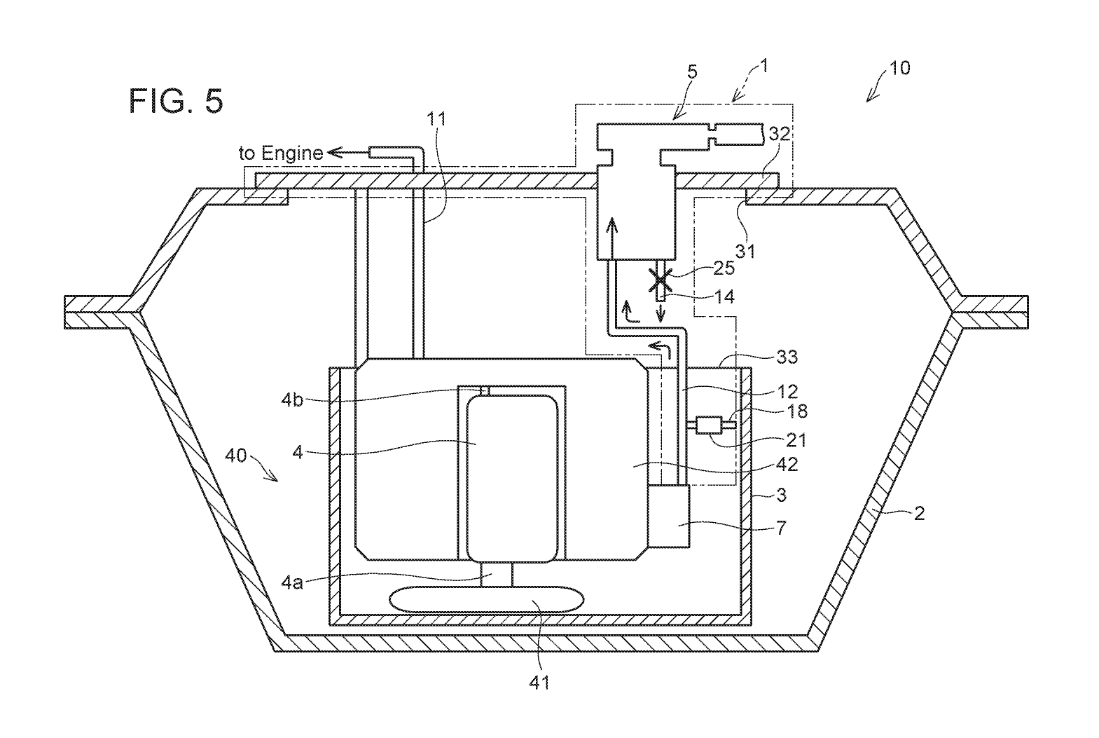

Further, the guiding line 12 is not limited in configuration to the embodiment. In a sensor device 1 according to another embodiment, as shown in FIG. 5, a branch line 18 branches from the guiding line 12. The branch line 18 branches from the guiding line 12 on an upstream side of the detection electrodes 61. A portion of the fuel flowing through the guiding line 12 flows into the branch line 18. An end of the branch line 18 opens inside of the reserve cup 3. The branch line 18 is provided with a valve 21 (which is an example of a relief mechanism). The valve 21 opens and closes the branch line 18. When the valve 21 opens, the branch line 18 opens. When the branch line 18 opens, a portion of the fuel flowing through the guiding line 12 flows through the branch line 18 to be discharged into the reserve cup 3. Thus, the valve 21 is configured to allow fuel to flow out from the fuel passage on the upstream side of the detection electrodes 61 (i.e. from the inside of the guiding line 12). It should be noted that while the valve 21 was used as an example of the relief mechanism in the embodiment shown in FIG. 5, without being limited to this configuration, the valve 21 may be replaced by a throttle (not illustrated).

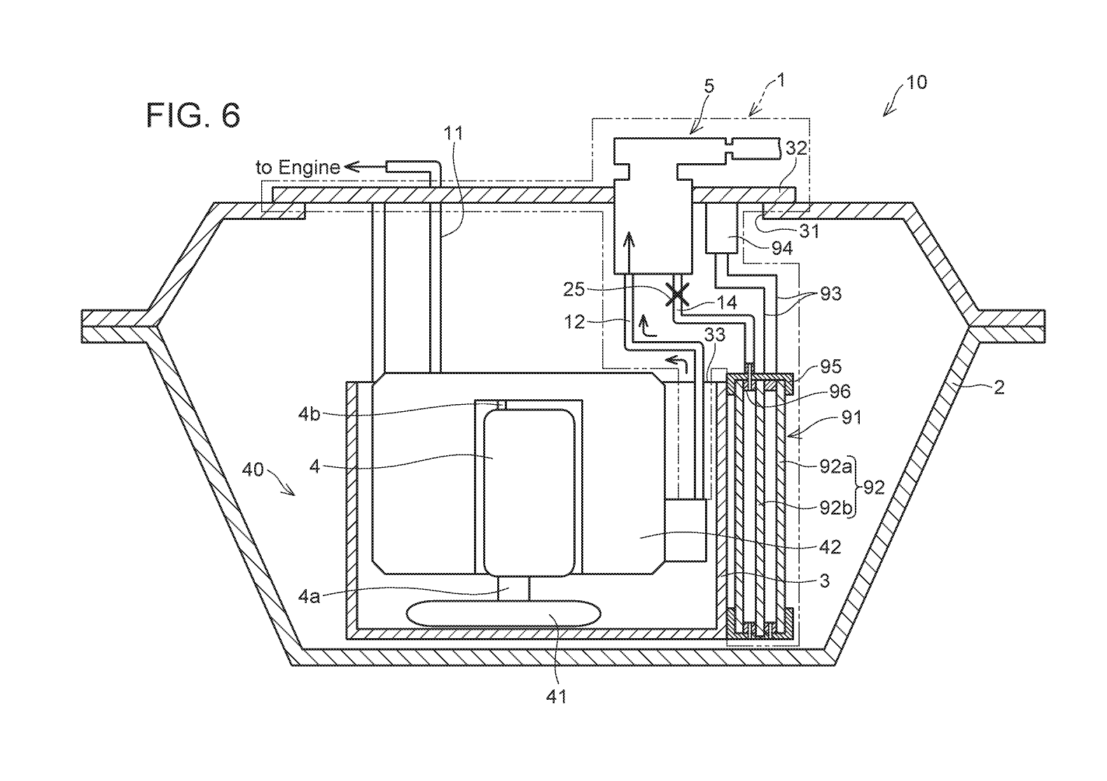

Further, while the discharge line 14 opened toward the inside of the reserve cup 3 in the embodiment, the discharge line 14 is not limited to this configuration. In a sensor device 1 according to another embodiment, as shown in FIG. 6, the discharge line 14 is connected to a storage case 91 (which is an example of the second case). The storage case 91 is configured to store fuel therein to detect a liquid level (which is an example of a second property) of the fuel housed in the fuel tank 2. The storage case 91 comprises a pair of electrodes 92 and caps 95. One electrode 92a is disposed outside, and the other electrode 92b is disposed inside. The outside electrode 92a surrounds the inside electrode 92b. The electrodes 92 are connected to a circuit (not illustrated) via a harness 93 and a connector 94. The caps 95 are fixed to both ends of the electrodes 92. The storage case 91 has a side wall formed by the electrodes 92. The cap 95 is provided with an introduction port 96 configured to introduce fuel into the storage case 91. The discharge line 14 (i.e., the fuel passage on the downstream side of the detection electrodes 61) communicates with an inside of the storage case 91. The discharge line 14 is connected to the introduction port 96. The fuel having flowed through the discharge line 14 is introduced into the storage case 91 via the introduction port 96. The area of the flow passage at the introduction port 96 is smaller than the flow passage area of the fuel passage on the upstream side of the introduction port 96. In other words, a diameter of the introduction port 96 is smaller than a diameter of the fuel passage on the upstream side of the introduction port 96. In the embodiment shown in FIG. 6, a first throttle (which is an example of the first resistance portion) is formed by the introduction port 96. The flow of fuel is obstructed by the introduction port 96 (first throttle).

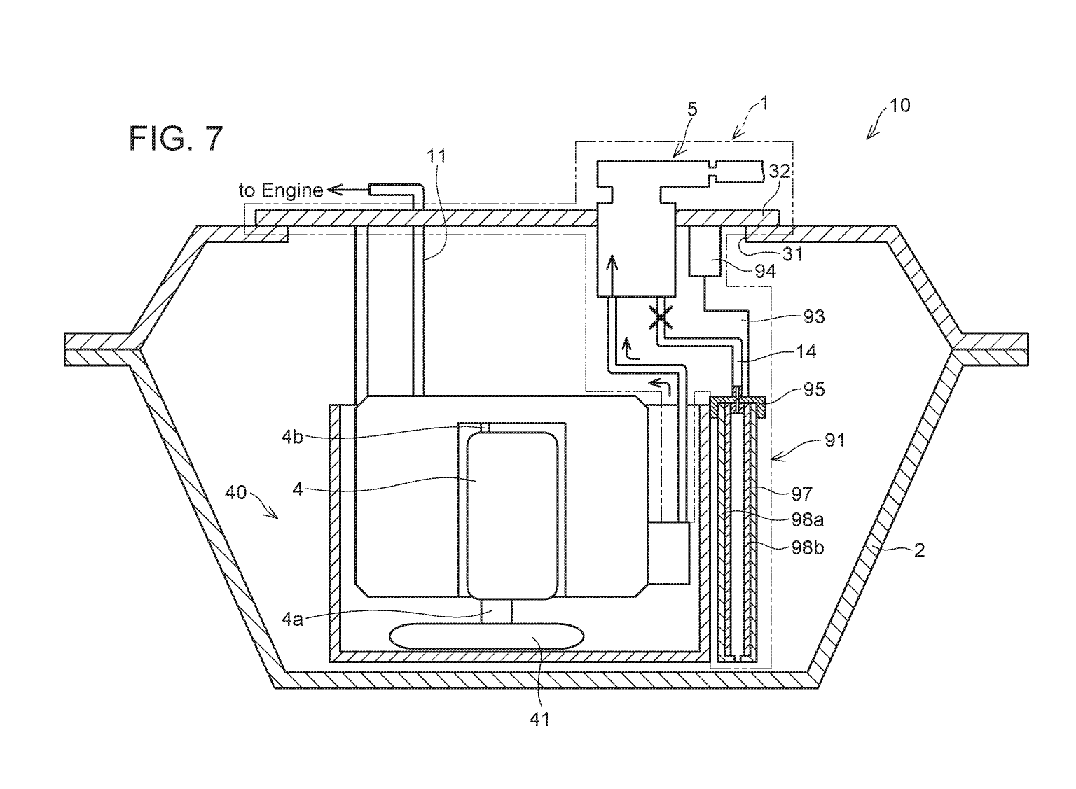

Further, the storage case 91 is not limited in configuration to the embodiment. In another embodiment, as shown in FIG. 7, the storage case 91 comprises a side wall 97 and a pair of electrodes 98a and 98b. Components shown in FIG. 7 that are identical to those shown in FIG. 6 are given the same signs, and descriptions thereof will be omitted. The side wall 97 is in the shape of a cylinder. The caps 95 are fixed to both ends of the side wall 97. The pair of electrodes 98a and 98b are in the shape of flat plates, and are fixed to the side wall 97. The electrodes 98a and 98b face each other, and fuel is introduced into a space between the pair of electrodes 98a and 98b.

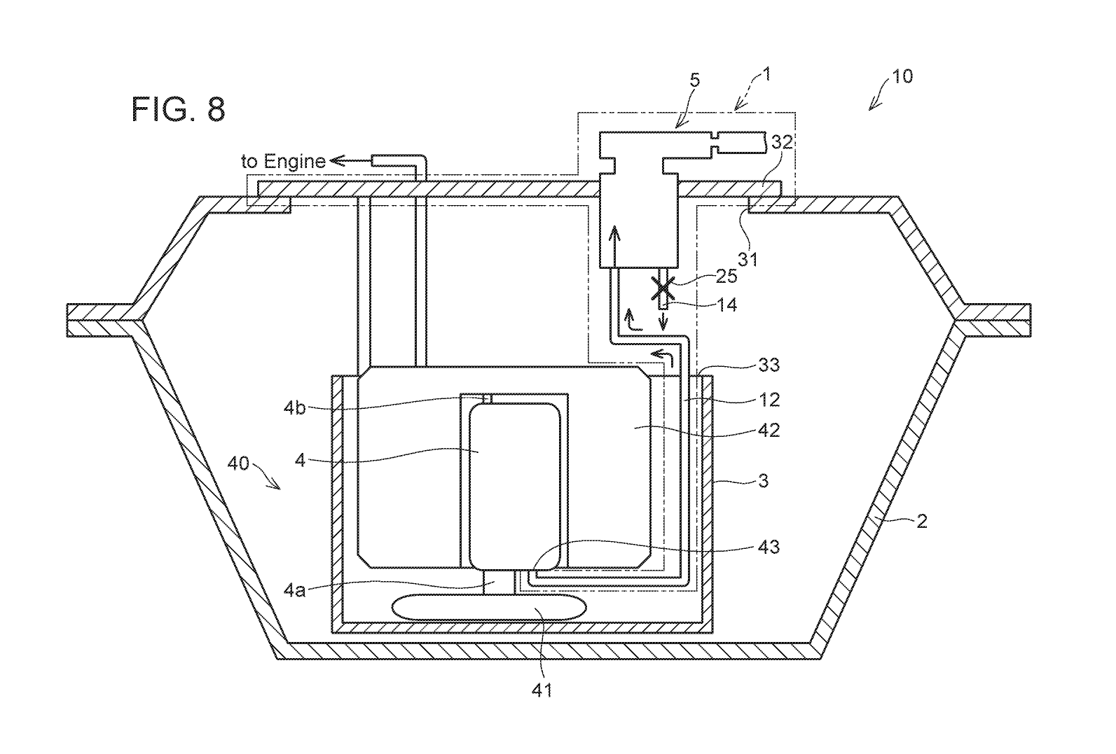

Further, while the guiding line 12 had one end connected to the pressure regulator 7 in the embodiment, the guiding line 12 is not limited to this configuration. In a sensor device 1 according to another embodiment, as shown in FIG. 8, the guiding line 12 has one end connected to a vapor jet 43. The vapor jet 43 causes vapor generated in the fuel pump 4 to be discharged out of the fuel pump 4. Fuel pressurized by the fuel pump 4 is ejected from the vapor jet 43 to the guiding line 12 together with the vapor.

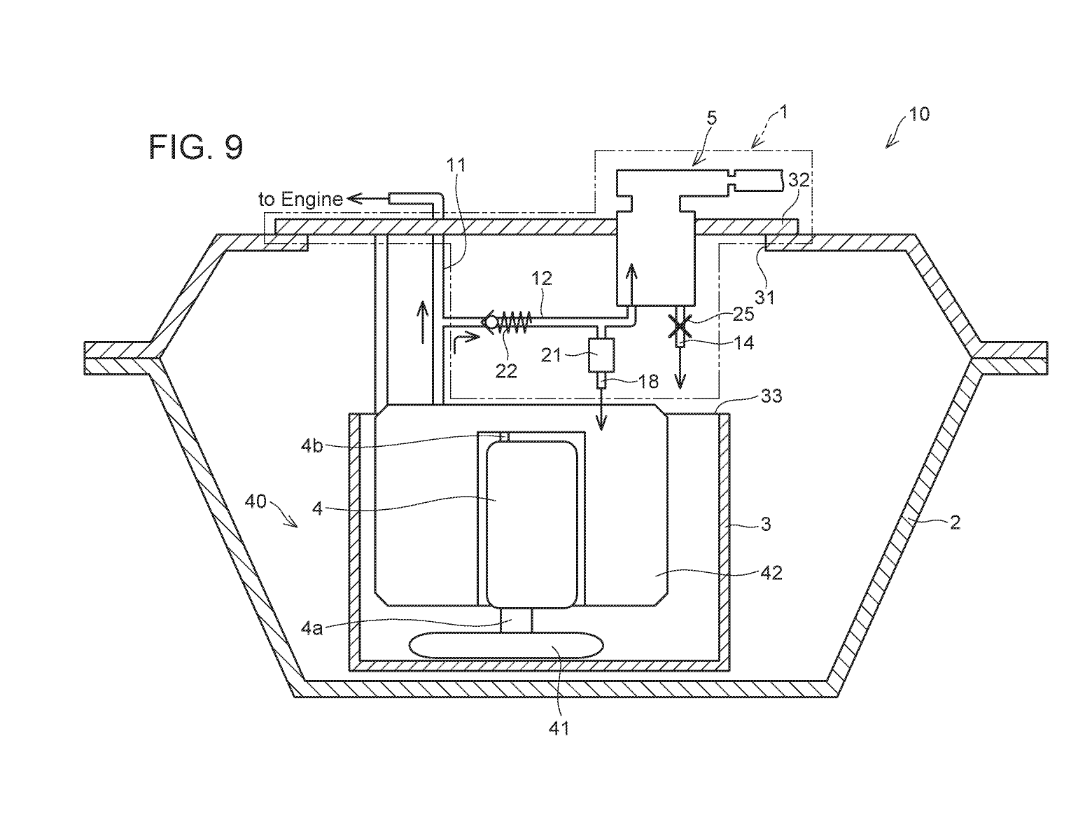

Further, in a sensor device 1 according to still another embodiment, as shown in FIG. 9, the guiding line 12 has one end connected to the supply line 11. That is, the guiding line 12 branches from the supply line 11. A portion of the fuel flowing through the supply line 11 flows into the guiding line 12. The guiding line 12 is provided with a residual pressure holding valve 22. The residual pressure holding valve 22 opens when the pressure of fuel in the guiding line 12 on a supply line 11 side of the residual pressure holding valve 22 becomes equal to or higher than a predetermined pressure, and closes when the pressure becomes equal to or lower than the predetermined pressure. Due to this, the residual pressure holding valve 22 holds the pressure of fuel on the supply line 11 side equal to or higher than the predetermined pressure. Therefore, the pressure of fuel flowing through the supply line 11 is maintained, so that the high-pressure fuel is sent to the engine of the vehicle. Further, the branch line 18 branches from the guiding line 12. The branch line 18 is provided with the valve 21 (which is an example of the relief mechanism). As a relief mechanism, the valve 21 may be replaced by a throttle (not illustrated).

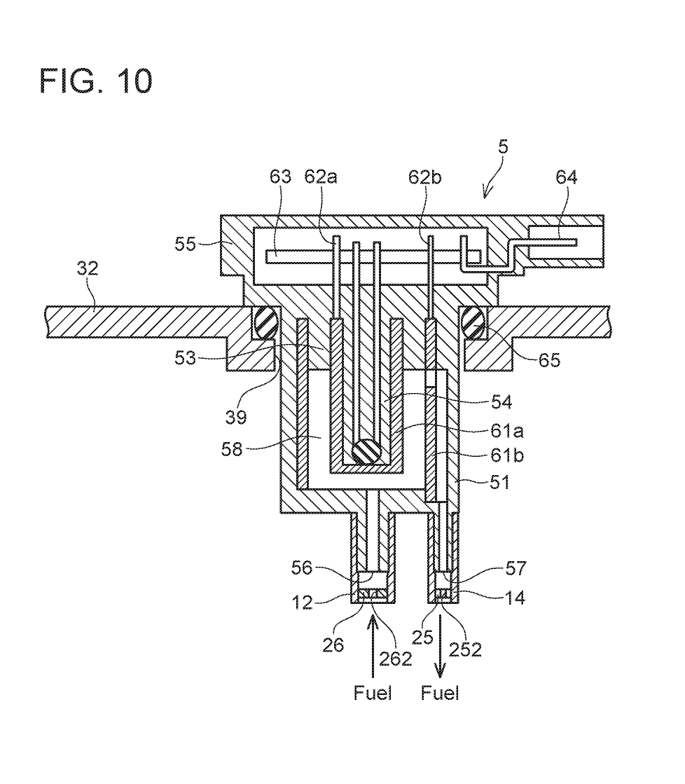

Further, while the lower case 51 and the upper case 55 were formed separately from each other in the embodiment, the lower case 51 and the upper case 55 are not limited to this configuration. In a fuel property sensor 5 according to still another embodiment, as shown in FIG. 10, the lower case 51 and the upper case 55 are formed integrally with each other. Further, while the lower case 51 and the set plate 32 were formed integrally with each other in the above embodiment, the lower case 51 and the set plate 32 are not limited to this configuration. In the fuel property sensor 5 according to still another embodiment, as shown in FIG. 10, the lower case 51 and the set plate 32 are formed separately from each other. The lower case 51 is inserted in an opening 39 formed in the set plate 32. This causes the fuel property sensor 5 to be attached to the set plate 32. The throttle 25 is disposed in the discharge line 14, and the throttle 26 is disposed in the guiding line 12. Notably, as in the embodiment shown in FIG. 4, the throttles 25 and 26 may be replaced by a first throttle (which is an example of the first resistance portion) formed by a discharge port arranged in the lower case 51 and a second throttle (which is an example of the second resistance portion) formed by an introduction port arranged in the lower case 51, respectively (both not illustrated).

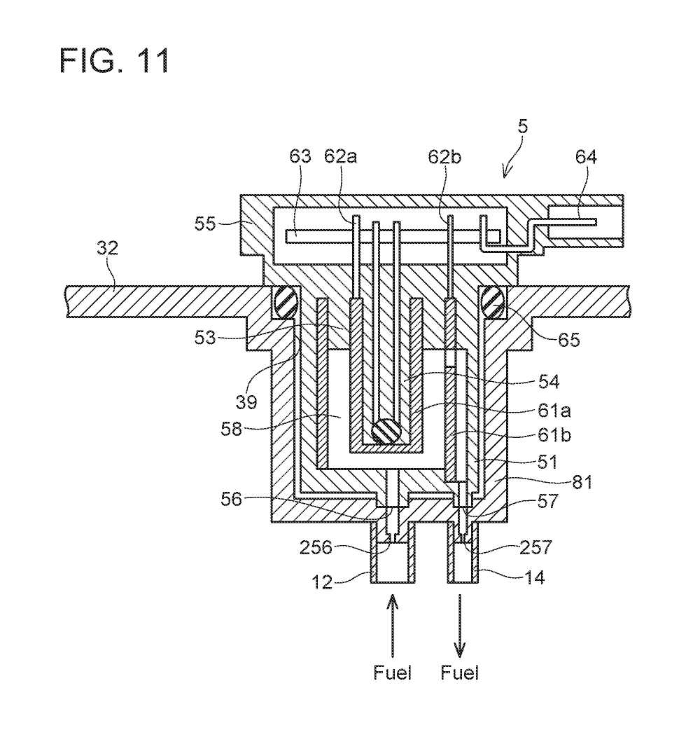

Further, the sensor device is not limited in configuration to the embodiment. As shown in FIG. 11, a sensor device according to still another embodiment further comprises a cover case 81 covering the lower case 51 (which is an example of the first case). The cover case 81 houses the lower case 51. The cover case 81 covers the lower case 51 from below and surrounds a bottom part and a side wall of the lower case 51. The cover case 81 is fixed to and integrated with the set plate 32. Formed in a bottom part of the cover case 81 is a discharge port 257 (which is an example of a second discharge port) configured to discharge fuel to the outside. The discharge port 257 of the cover case 81 communicates with the discharge port 57 (which is an example of a first discharge port) of the lower case 51. The discharge line 14 is connected to the discharge port 257 so that fuel is discharged into the discharge line 14 via the discharge port 57 of the lower case 51 and the discharge port 257 of the cover case 81. The discharge port 257 provided in the cover case 81 forms a first throttle (which is an example of the first resistance portion). The area of the flow passage at the discharge port 257 is smaller than the flow passage areas of the fuel passage on upstream and downstream sides of the discharge port 257. In other words, a diameter of the discharge port 257 is smaller than diameters of the fuel passage on the upstream and downstream sides of the discharge port 257. The flow of fuel is obstructed by the discharge port 257 (first throttle).

Further provided in the bottom part of the cover case 81 is an introduction port 256 (which is an example of a second introduction port) through which fuel is introduced into the housing space 58. The introduction port 256 of the cover case 81 communicates with the introduction port 56 (which is an example of a first introduction port) of the lower case 51. The guiding line 12 is connected to the introduction port 256 so that fuel is introduced from the guiding line 12 into the housing space 58 via the introduction port 256 of the cover case 81 and the introduction port 56 of the lower case 51. The introduction port 256 provided in the cover case 81 forms a second throttle (which is an example of the second resistance portion). The area of the flow passage at the introduction port 256 is smaller than the flow passage areas of the fuel passage on upstream and downstream sides of the introduction port 256. In other words, a diameter of the introduction port 256 is smaller than diameters of the fuel passage on the upstream and downstream sides of the introduction port 256. The flow of fuel is obstructed by the introduction port 256 (second throttle).

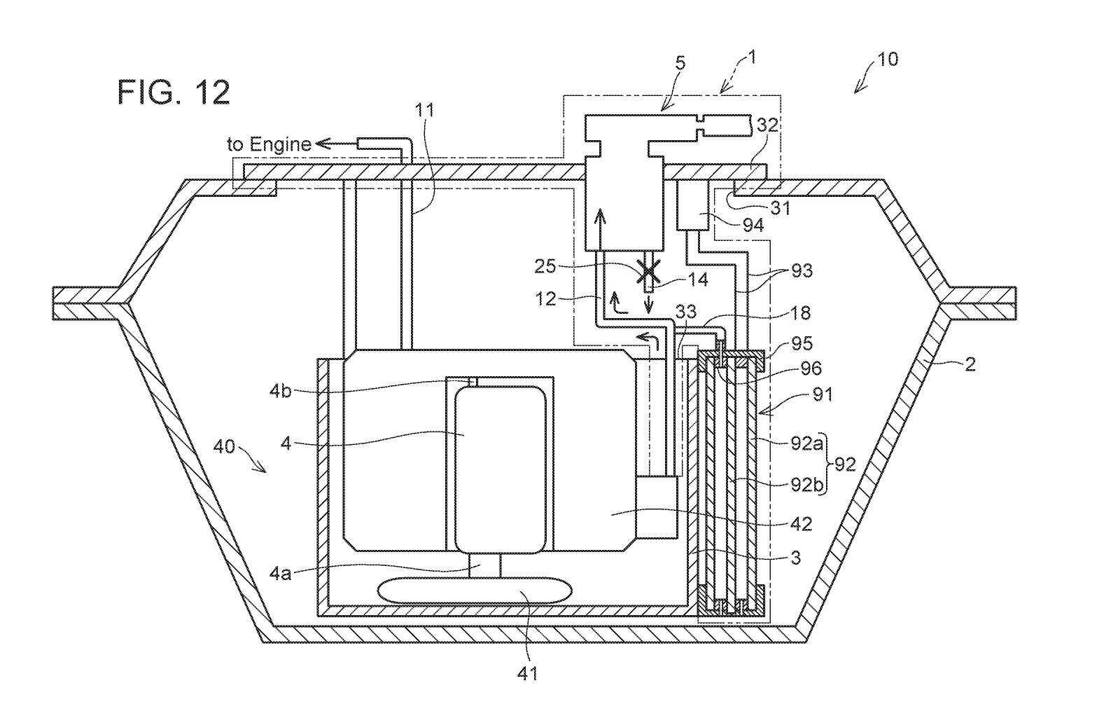

Further, while the end of the branch line 18 opened inside of the reserve cup 3 in the embodiment shown in FIG. 5, the distal end of the branch line 18 is not limited to this configuration. Further, while the discharge line 14 was connected to the storage case 91 in the embodiment shown in FIG. 6, the discharge line 14 is not limited to this configuration. In another embodiment, as shown in FIG. 12, the end of the branch line 18 (which is an example of a branch passage) may be connected to the storage case 91. The branch line 18 branches from the guiding line 12. The branch line 18 branches from the guiding line 12 on the upstream side of the detection electrodes 61 (not illustrated in FIG. 12) in the fuel property sensor 5. A portion of the fuel flowing through the guiding line 12 flows into the branch line 18. The branch line 18 communicates with the inside of the storage case 91. The branch line 18 is connected to the introduction port 96. The fuel having flowed through the branch line 18 is introduced into the storage case 91 through the introduction port 96. The area of the flow passage at the introduction port 96 is smaller than the flow passage area of the branch passage on an upstream side of the introduction port 96. In other words, a diameter of the introduction port 96 is smaller than a diameter of the branch passage on the upstream side of the introduction port 96. In the embodiment shown in FIG. 12, the introduction port 96 forms a third throttle (which is an example of a third resistance portion). The flow of fuel is obstructed by the introduction port 96 (third throttle).

Specific examples of the present teachings have been described in detail, however, these are mere exemplary indications and thus do not limit the scope of the claims. The art described in the claims includes modifications and variations of the specific examples presented above. Technical features described in the description and the drawings may technically be useful alone or in various combinations, and are not limited to the combinations as originally claimed. Further, the art described in the description and the drawings may concurrently achieve a plurality of aims, and technical significance thereof resides in achieving any one of such aims.

### REFERENCE SIGNS LIST

1: Sensor device
2: Fuel tank
3: Reserve cup
4: Fuel pump
4a: Suction port
4b: Ejection port
5: Fuel property sensor
7: Pressure regulator
10: Fuel supply unit
11: Supply line
12: Guiding line
14: Discharge line
18: Branch line
21: Valve
22: Residual pressure holding valve
25: Throttle
25: Throttle
27: Filter
28: Filter
31: Opening
32: Set plate
33: Opening
39: Opening
40: Pump unit
41: Suction filter
42: High-pressure filter
43: Vapor jet
51: Lower case
53: Lid portion
54: Protruding portion
55: Upper case
56: Introduction port
57: Discharge port
58: Housing space
59: Opening
61: Detection electrode
62: Internal terminal
63: Circuit portion
64: External terminal
65: Sealing material
81: Cover case
91: Storage case
92: Electrode
92a: Outside electrode
92b: Inside electrode
93: Harness
94: Connector
95: Cap
96: Introduction port
97: Side wall
98: Electrode
156: Introduction port
157: Discharge port
252: Communication hole
256: Introduction port
257: Discharge port
262: Communication hole

1. A sensor device comprising:
a fuel passage through which fuel ejected from a fuel pump in a fuel tank flows; and
a detection electrode configured to make contact with fuel flowing through the fuel passage to detect a first property of fuel,
wherein
a first resistance portion configured to resist a flow of fuel on a downstream side of the detection electrode is provided in the fuel passage.

2. The sensor device according to claim 1, wherein
a second resistance portion configured to resist the flow of fuel on an upstream side of the detection electrode is provided in the fuel passage, and
a resistance of the first resistance portion to the flow of fuel is greater than a resistance of the second resistance portion to the flow of fuel.

3. The sensor device according to claim 1, further comprising:
a set plate attached to the fuel tank;
a first case housing the detection electrode and forming a part of the fuel passage; and
a cover case formed integrally with the set plate, and covering the first case,
wherein
a first discharge port configured to discharge fuel in the first case to outside is provided in the first case,
a second discharge port communicating with the first discharge port of the first case is provided in the cover case, and
the first resistance portion is formed by the second discharge port.

4. The sensor device according to claim 1, further comprising:
a set plate attached to the fuel tank; and
a first case housing the detection electrode and forming a part of the fuel passage;
wherein
a discharge port configured to discharge fuel in the first case to outside is provided in the first case, and
the first resistance portion is formed by the discharge port.

5. The sensor device according to claim 1, further comprising
a second case configured to store fuel therein to detect a second property of fuel,
wherein
the fuel passage communicates with an inside of the second case on a downstream side of the detection electrode.

6. The sensor device according to claim 5, wherein
an introduction port configured to introduce fuel into the second case is provided in the second case, and
the first resistance portion is formed by the introduction port.

7. The sensor device according to claim 1, further comprising:
a second case configured to store fuel therein to detect a second property of fuel, and
a branch passage branching from the fuel passage on an upstream side of the detection electrode, and communicating with an inside of the second case.

8. The sensor device according to claim 7, wherein
an introduction port configured to introduce fuel into the second case is provided in the second case, and
a third resistance portion is formed by the introduction port.

9. The sensor device according to claim 1, further comprising
a relief mechanism configured to allow fuel to flow out of the fuel passage on an upstream side of the detection electrode.

10. The sensor device according to claim 1, wherein
the first resistance portion is formed by a throttle or a filter arranged in the fuel passage.
